(19) United States
(12) Patent Application Publication
(10) Pub. No.: US 2007/0243232 A1
(43) Pub. Date: Oct. 18, 2007

(54) Method to treat and prevent asthma attacks using throat lozenges and orally-retained liquids containing magnesium

(76) Inventor: George Andrew Eby III, Austin, TX (US)

Correspondence Address: George Andrew;Eby III, 14909-C Fitzhugh Road, Austin, TX, 78736, US

(21) Appl. No.: 11/279,636

(22) Filed: Apr. 13, 2006

(51) Int. Cl. A61K 33/14; A61K 47/02

(52) U.S. Cl. 424/439; 424/440; 424/681

(57) ABSTRACT

Asthma is an inflammatory disease of the airways in the lungs and bronchial tubes, the lower airway, that impairs breathing, causes wheezing, coughing and excess mucus and phlegm production. Asthma is the source of about one-fourth of all emergency room admissions. Throat lozenges and orally-retained liquids, such as syrups, containing magnesium are used in the invention to rapidly terminate asthma attacks and prevent asthma attacks. Magnesium lozenges and magnesium orally-retained liquids are effective in asthma rescue. Treatment of asthma with orally-retained magnesium, an essential human nutrient, is much safer than treatment by drugs yet they appear as effective as current drugs. An added benefit from magnesium treatment is relaxation. A preferred composition is 100 mg of magnesium from 400 mg magnesium chloride in a 4-gram lozenge. Magnesium lozenges or orally-retained liquids are administered as needed to treat asthma attacks or prevent incipient asthma attacks. Throat lozenges and orally-retained liquids of any pharmaceutically acceptable composition containing any pharmaceutically acceptable compound of magnesium are disclosed and claimed as effective in the treatment and prevention of asthma.

### REFERENCES CITED

US patents: U.S. Patent Application 20050196434, U.S. Pat. No. 5,898,037

Foreign patents: none

Other references: Blitz M et al. Aerosolized magnesium sulfate for acute asthma: a systematic review. Chest. July 2005;128(1):337-44.

Rowe B H, et al. Intravenous magnesium sulfate treatment for acute asthma in the emergency department: a systematic review of the literature. Annals of Emergency Medicine. September 2000;36(3):181-90.

Tong G M, Rude R K Magnesium deficiency in critical illness. Journal of Intensive Care Med. January-February 2005;20(1):3-17.

### GOVERNMENT INTERESTS

### STATEMENT REGARDING FED SPONSORED R & D

This invention was not supported or associated with any federally sponsored research and/or development project or funds.

### DESCRIPTION

### BACKGROUND OF THE INVENTION

Asthma is characterized by inflammation of the air passages resulting in the temporary narrowing of the airways that transport air from the nose and mouth into the lungs. Asthma symptoms can be caused by allergens or irritants that are inhaled into the lungs, resulting in inflamed, plugged and constricted airways. Some asthma is intrinsic, meaning that other causes, such as cold air, is causative. Symptoms include difficulty breathing, excessive mucus and phlegm production, wheezing, coughing and tightness in the chest. In the United States, each day about 30,000 people have an asthma attack, with about 5,000 of them requiring emergency room attention with about 14 deaths per day. Asthma accounts for about one-fourth of all emergency room admissions. One in 15 Americans has asthma, with about one half of all asthma cases being caused by allergic reactions. The prevalence of asthma is increasing. It is the most chronic condition for American children affecting 7 to 10% of them, while asthma affects adults by about one half that incidence. Incidence is correlated with poverty, air quality and allergens and malnutrition. Cost of asthma is about $18 billion per year, with $10 billion being for hospitalization and $5 billion in indirect costs for illness and death. Asthma is a leading cause of work absenteeism and impaired work performance.

Many rescue treatments for asthma exist which include drugs in inhaled form and in pill form. Asthma medications inhaled into the lungs are used to rescue the patient from an asthma attack. They include beta-agonists such as short- and long-acting bronchodilators, corticosteroids, cromolyn, nedocromil and combinations. Pill forms include leukotriene modifiers and theophylline. Each of these drugs has its place in the treatment of asthma. However, most if not all, have side effects that can make long-term treatment problematic without careful medical supervision and treatment of the side effects, including termination of specific drug treatment.

Magnesium has been used in inhaled forms and in injections to treat asthma, rescuing the patient. However, no evidence that magnesium throat lozenges or orally-retained magnesium liquids have been used to treat asthma was found. There is no evidence found that any anti-asthma medication has been prepared or used in lozenge or orally-retained forms. Orally-retained magnesium compositions include solids and liquids used in the mouth and throat where the pharmacological benefit of magnesium is applied in the mouth and throat.

### PRIOR OR RELATED ART

Magnesium throat lozenges for treatment of asthma are not obvious from the prior or related art because the improvement in response is very large, very fast, unexpected and currently unexplained relative to the prior art. Magnesium has been used in the prior art to treat asthma.

Transdermal delivery of magnesium for the treatment of asthma (U.S. Patent Application 20050196434), magnesium inhalants (U.S. Pat. No. 5,898,037 and Blitz M et al. Aerosolized magnesium sulfate for acute asthma: a systematic review. Chest. July; 2005;128(1):337-44.) and magnesium injections (Rowe B H, et al. Intravenous magnesium sulfate treatment for acute asthma in the emergency department: a systematic review of the literature. Annals of Emergency Medicine. September 2000;36(3):181-90.) to treat asthma are also known in the prior art. Dietary deficiencies of magnesium are correlated with asthma attacks (Tong G M, Rude R K Magnesium deficiency in critical illness. Journal of Intensive Care Med. January-February 2005;20(1):3-17.), however according to Tong and Rude, magnesium therapy has been shown useful only in those people with very severe exacerbations. Thus they concluded that critically ill asthmatic patients as a group are most likely to benefit from empirical magnesium therapy,

This invention corrects the errors of the past because this invention is shown to be useful in mild asthma, moderate asthma and severe asthma as is shown in the Examples and is anticipated to be fully functional in the critically ill. With the present invention there is no requirement for the person in need of treatment to be “critically ill”. No mention of magnesium being used in the mouth or throat to treat asthma was found in either the medical literature (Index Medicus) online search engines (Google and Yahoo) or prior or related art. No mention of magnesium lozenges or orally-retained liquids in the treatment of asthma in the prior or related art was found. Perhaps this is because magnesium from magnesium lozenges or orally-retained liquids does not flow into the lower airway (trachea, bronchial tubes and lungs) where it could be expected to perform a useful function in the treatment of the lung and bronchial disorder, asthma. Consequently, there is no evident contact by magnesium from lozenges or orally-retained liquids with the lungs or lower airway as is required for other drugs to effect rapid rescue from asthma symptoms. Thus, the invention is inobvious and it is not known in the prior or related art. A number of studies of acute bronchial asthma have shown that intravenous or nebulized (inhaled) magnesium sulfate may improve symptoms over a course of hours, but not seconds and minutes as has been found using the present invention. Upon treatment over several weeks with oral (swallowed) magnesium, a significant decrease in symptom frequency results. Injections of magnesium (magnesium sulfate) in the 200 mg range provided near instant increases in blood levels of magnesium which are modestly effective in treating asthma and effecting a rescue. Inhalants are used to apply magnesium (usually from magnesium sulfate) directly into the lungs and bronchial tissues combined with other drugs, however there is debate as to the efficacy of this treatment since there is little difference—if any—between responses to those drugs used with and without magnesium.

### SUMMARY OF THE INVENTION

The present invention discloses an improvement in the use of swallowed, injected and inhaled magnesium to treat acute asthma symptoms, and an improvement over most asthma drugs in safety in the treatment of asthma. Magnesium is believed safer to use to treat asthma than pharmaceutical drugs. Orally-retained compositions such as throat lozenges and orally-retained liquids containing magnesium compounds are envisioned, disclosed and claimed as being effective rescue treatments for acute asthma attacks. Magnesium throat lozenges, when allowed to dissolve in the mouth are found to be effective in the treatment of asthma, thus rescuing the asthma sufferer. Any compound of magnesium when used in the lozenge-form rapidly terminates asthma symptoms effecting a rescue. Magnesium throat lozenges and orally-retained liquids are an improvement over magnesium injections and magnesium inhalants and swallowed magnesium tablets because of their vastly stronger efficacy, simplicity of use, lack of need for hospitalization, safety of use, ready availability, over-the-counter status and very low cost in the treatment and prevention of asthma. Both solids and liquids containing magnesium for sustained retention in the mouth to treat or effect a rescue from asthma or prevent asthma are also envisioned, disclosed and claimed.

### DETAILED DESCRIPTION

The present invention discloses an improvement in the use of magnesium to treat asthma. Magnesium injections and magnesium inhalants have been used to treat asthma, and this invention is an improvement to those earlier means of magnesium delivery since orally-retained lozenges and orally-retained liquids containing magnesium are convenient, safe, effective and inexpensive. No injections, inhalations, physicians or hospital treatment are required to administer this treatment, although there is nothing envisioned to preclude the use of this treatment by physicians or hospitals.

Throat lozenges comprising magnesium compounds are described as being an effective treatment and preventative for asthma. Throat lozenges are equivalent in function to compressed tablets, hard candies, troches, discs, tablets, soft candies, pastilles, sub-lingual or buccal tablets and other solid objects held in the mouth and dissolved in the mouth, whether they are sweet or not sweet, fast or slow dissolving, and all are envisioned, disclosed and claimed. Sweetness is not a required property of magnesium throat lozenges, but it is desired. Magnesium throat lozenges, when allowed to dissolve in the mouth, are found to be effective in the treatment and prevention of asthma, effective against the symptoms of asthma such as broncoconstriction, phlegm and mucus production, mucus plugs in the lungs and bronchial and chest allergic reactions and in intrinsic asthma. Inflammation in the lungs and lower airways resulting in phlegm, mucus and blockage in the lungs and lower air-ways is also terminated using orally-retained magnesium such as results from use of lozenges containing magnesium. Asthma can be prevented by treating with magnesium lozenges and orally-retained liquids as needed. If an asthma attack is felt to come on, immediate use of one or more magnesium lozenges or a composition of an orally-retained liquid either prevents the attack, reduces its intensity, or rapidly terminates the attack. Repeated treatment may be needed, which reduces or prevents additional asthma attacks.

Orally-retained liquids containing pharmaceutically acceptable compound(s) of magnesium of any chemical and physical composition are useful and are envisioned, disclosed and claimed for the prevention and treatment of asthma as a rescue medication or for the prevention of asthma and asthma symptoms. Magnesium administered in the form of sublingual liquid drops, gargles, syrups, mouthwashes, throat sprays and other liquids containing magnesium compounds are also envisioned, disclosed and claimed. Their function is essentially identical to throat lozenges, although they are not solids, but are liquid or nearly liquid in some fashion. The ingredients and procedures for making orally-retained liquids for use in the mouth and throat are in the prior art and are well known and are directly applicable to making magnesium orally-retained liquids for mouth and throat use. Dextrose, sugar, corn syrup, maltose, fructose and sucrose are all common lozenge and syrup ingredients and are used as needed.

Magnesium is a natural antihistamine required for proper function of the immune system, and some published evidence shows that increased dietary intake of magnesium reduces the incidence of asthma. Magnesium naturally decreases the uptake of calcium by bronchial smooth muscle cells, which in turn leads to bronchodilation. Magnesium may also have a role in inhibiting mast cell degranulation, thus reducing inflammatory mediators such as histamine, thromboxanes, and leukotrienes. In addition, magnesium inhibits the release of acetylcholine from motor nerve terminals and depresses the excitability of muscle fiber membranes. However, there is no indication as to how throat lozenges or orally-retained liquids containing magnesium affects the lung tissue is such a rapid and powerful manner as to allow these compositions to rapidly, within seconds and minutes, and definitely not within hours, days or longer, terminate an episode of asthma and prevent asthma.

There is no requirement for sweetness or flavor-masking for magnesium lozenges, although such are envisioned and described. There are no problems with flavor masking because magnesium compounds are not particularly objectionable or astringent in taste. Stevia is an outstanding sweetener for magnesium lozenges where extra sweetness is desired and it has a mild flavor-masking effect. Formulations to be held in the mouth may also desirably contain flavoring agents such as, for example but not limited to, anise, anethole, eucalyptol, wintergreen, licorice, clove, cinnamon, spearmint, cherry, lemon, orange, lime, menthol, peppermint and various combinations thereof, each of which has its own flavor-masking effect.

A lozenge or liquid composition formula that allows a 1 to 10 ratio of the magnesium compound and composition base can be used to make homeopathic lozenges under the U. S. Federal Food, Drug, and Cosmetic Act as a 1× titration. An example is 400 mg of magnesium chloride (100 mg elemental magnesium) in a 4 gram sugar and corn syrup lozenge or 4 gram compressed dextrose lozenge.

Swallowed magnesium tablets or pills are not known to be used to effect rescues for patients from asthma, but swallowing magnesium pills has been used in attempts to provide nutritional support to prevent asthma but without any rescue capability, and there is a belief that dietary deficiency of magnesium is implicated in causing asthma. However, about 70% of the American public is deficient in magnesium, but only about 6% of Americans have asthma, consequently there is more to the issue of asthma than dietary magnesium deficiency. In example 7 below, 500 mg of magnesium as a dietary supplement had little or no effect on frequency or severity of asthma symptoms, but 500 mg of magnesium as throat lozenges prevented asthma symptoms, thus showing that it is not the amount of magnesium ingested per day, but the means of ingesting magnesium that is important in treating and preventing asthma.

Dosages of any amount of magnesium are anticipated for use in throat lozenges and orally-retained liquids to effectively and safely treat asthma in people needing treatment. Preference is given to doses between 0.001 and 10,000 mg of magnesium per lozenge, and greater preference is given to lozenges in the 1 to 500 mg range, while even greater preference is given to lozenges in the 50 to 200 mg range. The most effective dose has not been established, but 100 mg doses are effective, safe, pleasant, useful and convenient.

All pharmaceutically acceptable compounds of magnesium are anticipated, disclosed and claimed in this invention. The more ionized the magnesium compound, the more effective will be the treatment and preferred the composition. The most ionized magnesium compounds are believed to be—in descending order—magnesium chloride, magnesium acetate, magnesium gluconate, magnesium lactate, magnesium malate, magnesium glutamate, magnesium aspartate, magnesium citrate, magnesium glycinate, magnesium carbonate, magnesium taurinate, magnesium orotate, magnesium hydroxide, magnesium lysinate, magnesium adipate, magnesium bicarbonate and magnesium oxide. Strong preference is also given to homeopathic magnesium chloride (Magnesia Muriatica), magnesium carbonate (Magnesia Carbonica), magnesium phosphate (Magnesia Phosphorica), and magnesium sulfate (Magnesia Sulphuric) because their record of safety in other homeopathic medicines has been long established. Magnesium chloride is the most preferred compound for this invention because it is highly ionizable, has a pleasant taste when diluted in a homeopathic lozenge preparation at 1× titration, and it is effective and safe. Magnesium glycinate is also highly preferred because it is naturally sweet and it is effective and safe.

A suitable 4-gram magnesium chloride (100 mg magnesium) throat lozenge formulation for direct compression comprises 400 mg magnesium chloride, 8 mg stevia leaf extract, 20 mg silicon, 100 mg glycerol monostearate, 9 mg peppermint oil, and 3463 mg agglomerated dextrose. A suitable 4-gram magnesium chloride (100 mg magnesium) hard candy throat lozenge formulation composition comprises 400 mg magnesium chloride, 8 mg stevia leaf extract, 9 mg peppermint oil and 3583 mg of hard candy base comprised of sucrose and corn syrup, boiled to a hard candy.

The United States Recommended Dietary Allowance (RDA) for magnesium is 400 mg per day for adults. Most Americans ingest less than the RDA of magnesium. Since magnesium is an essential human nutrient, it is inherently safe to use in throat lozenges for asthma and it has no known side effects at effective doses less than a total of 600 to 800 mg total per day. Upon treatment over several weeks with oral (swallowed) magnesium, a significant decrease in asthma symptom frequency results. Higher total daily doses may cause loose bowels, and much higher doses may cause diarrhea and overdose. Magnesium throat lozenges are anticipated to be used in conjunction with other asthma medications as may be required, or they may be used by themselves with no other asthma treatment dependent upon the user's response to magnesium lozenge treatment and physician and patient requirements. The most common side effect of excess magnesium is diarrhea.

Although the examples below are for adults, examples of smaller dosages for children and infants are understood, and anticipated to yield similar benefits perhaps using proportionately lower doses dependent upon weight and the RDA for children. Children's candy and lollipops with magnesium are envisioned, disclosed and claimed as appropriate treatment and preventative for asthma.

All means of treating asthma by use of orally-retained magnesium are envisioned, disclosed and claimed herein. For example, application of magnesium compounds intranasally by nasal application comprising nasal sprays, nasal powders, nasal dusts, nasal vapors, nasal liquids, nasal drops, nasal gels and nasal packings can also be used to treat and prevent asthma since effective doses of magnesium are also provided in the oral and throat fluids by these means, and they have direct application to treating and preventing asthma. It can readily be realized that any agent including magnesium introduced into the nose can appear in oral and throat fluids. The magnesium compound is preferably maintained in contact within the mouth and throat for a sufficient length of time that relief from asthma symptoms is achieved. Maintenance of contact may also be achieved by gargling a mouthwash containing magnesium for a suitable length of time. Maintenance of contact may also preferably be achieved by the slow dissolution in the mouth of a suitably sized lozenge containing a magnesium compound. Maintenance of contact in the mouth may also be achieved by dissolving suitably sized pastilles, drops, or sub-lingual or buccal tablets, or nasal applications containing magnesium. The suitable length of contact, and thus the time of gargling, size of lozenge or tablet, or number of pastilles, is readily ascertainable to one of skill in the arts of homeopathy, herbal, and/or clinical medicine. Further, as with the concentrations of active ingredients to be employed, the length of contact may differ with the characteristics of the subject and the object(s) to be achieved.

Further, all anti-asthma drugs, comprising beta-agonists such as short- and long-acting bronchodilators, corticosteroids, cromolyn, nedocromil, leukotriene modifiers and theophylline and combinations in suitable dosage for throat lozenge use or in a orally-retained liquid form are herein anticipated and disclosed as effective against asthma, asthma symptoms, and chest congestion and as effective in the prevention of same. Orally-retained compositions comprising these asthma drug(s) with or without magnesium are herein anticipated and disclosed as effective against asthma, asthma symptoms and chest congestion and as effective in the prevention of same. The addition of these drugs will likely impair the safety of magnesium orally-retained compositions, but may improve their performance for severe, otherwise refractory asthmatic conditions.

Nutrients, such as vitamins, minerals, herbs and amino acids may be used with or without magnesium either singularly or in combination to effectively treat asthma when used in throat lozenge or orally-retained liquid form and such compositions and usages are anticipated and disclosed.

All publications and patent applications mentioned in this specification are herein incorporated by reference to the same extent as if each individual publication or patent application was specifically and individually indicated to be incorporated by reference.

In order to test the efficacy of the asthma attenuating effects of the present invention, the inventor conducted the following informal case studies, and their results are provided in the Examples below. In general, best results are obtained when treatment is commenced immediately when there is any suspicion of an asthma attack coming on although treatment is effective at any stage of an asthma attack. These examples are some of many that have resulted from use of magnesium lozenges in pre-clinical tests of their efficacy against asthma and chest congestion.

### EXAMPLES

### Example 1

Four hundred mg of magnesium chloride (100 mg magnesium) in a 4-gram hard candy (sucrose and corn syrup) lozenge prepared as a homeopathic remedy was used to treat intrinsic asthma in a 65 year old man. His asthma symptoms were initiated by cold air and cold feet. Awaking in the early morning hours and walking across a cold floor barefoot to the bathroom always caused immediate asthma with chest congestion, phlegm production and coughing, making resumption of sleep difficult. He was given magnesium chloride lozenges to keep on his bedstead, and he started to dissolve one upon awakening and prior to touching the cold floor. Even though the lozenge was not completely used by the time he walked to the bathroom, he did not develop asthma symptoms and was able to immediately return to sleep upon removing the lozenge from his mouth, perhaps leaving some dissolved magnesium in the mouth.

### Example 2

A single 4-gram lozenge having a dextrose, glyceryl monosterate, silica gel, peppermint oil and Stevia base containing 400 mg of magnesium chloride (100 mg elemental magnesium) was used to treat a sudden attack of acute asthma. The patient, a female in her early 40s, allowed the lozenge to dissolve in her mouth after finding that she had misplaced her prescription asthma inhaler. Before the lozenge had completely dissolved, the asthma attack, consisting of wheezing and extreme shortness of breath ended, and did not return that day—effecting a rescue. A further benefit noted was a feeling of relaxation, something that was not possible from use of her prescription rescue inhaler.

### Example 3

A single magnesium glycinate lozenge (100 mg magnesium) prepared to dissolve in 15 minutes in the mouth was used to treat severe, early morning coughing and lung mucus plug production from asthma. The treatment eliminated excess mucus production before the lozenge had completely dissolved effecting a rescue, and once existing mucus plugs were expectorated and related coughing terminated, symptoms did not return that day.

### Example 4

A 65-year old male used 4 magnesium malate (50 mg) lozenges periodically each day for one month to prevent asthma attacks. His incidences of asthma attacks requiring treatment declined steadily over that 1-month period, suggesting a long-term curative effect of magnesium lozenges.

### Example 5

A man used magnesium malate dietary supplement tablets (100 mg magnesium) as throat lozenges to prevent asthma and chest congestion. He used them 3 to 6 times per 24-hour day resulting in no asthma symptoms occurring while being treated.

### Example 6

A man used a syrup containing magnesium chloride in corn syrup and sugar (100 mg magnesium per teaspoon) to treat and prevent asthma. He allowed the syrup to remain in his mouth for 10 minutes. He used 4 teaspoons of the syrup per day to remain free of asthma symptoms.

### Example 7

A man had a 7 year history of use of swallowed magnesium glycinate and magnesium taurinate dietary supplements in the 500 mg magnesium daily range, which is more than the RDA for magnesium. He continued to experience severe chest congestion, slight wheezing, severe mucus production including mucus plugs with severe daily coughing while being treated with swallowed magnesium supplements. Upon switching to magnesium throat lozenges (100 mg magnesium from magnesium chloride) five times a day, the same dosage of magnesium previously swallowed, his asthma attacks stopped, his symptoms immediately came under control and he was able to prevent asthma symptoms while using magnesium throat lozenges.

### Example 8

A man used a magnesium glycinate dietary supplement (100 mg magnesium) as a throat lozenge with a 600 mg tablet of guaifenesin as a treatment for chest congestion and cough. Mucus production ceased and the treatment was effective.

### Example 9

A man used a magnesium chloride lozenge (100 mg magnesium) with prednisone early in the morning as a treatment for asthma, chest congestion and cough. Mucus production ceased and the treatment was effective providing relief for the entire day.

### Example 10

A man used a 400-mg magnesium oxide tablet as a throat lozenge (241.3 mg magnesium) early in the morning as a treatment for asthma, chest congestion and cough. The lozenges were beneficial in the treatment of his symptoms, but repeated doses were required, suggesting that magnesium oxide is not as bioavailable as other magnesium compounds.

I claim:

1. The method of treating asthma and chest congestion in people in need of treatment with throat lozenges comprising pharmaceutically acceptable magnesium.

2. The method of claim 1 wherein the magnesium compound is selected from the group comprising magnesium chloride, magnesium acetate, magnesium gluconate, magnesium lactate, magnesium malate, magnesium glutamate, magnesium aspartate, magnesium citrate, magnesium glycinate, magnesium carbonate, magnesium hydroxide, magnesium oxide, magnesium carbonate, magnesium phosphate, magnesium orotate, magnesium taurinate, magnesium lysinate, magnesium adipate, magnesium bicarbonate and magnesium sulfate and combinations of said magnesium compounds.

3. The method of claim 1 wherein the lozenge comprises a compressed tablet, hard candy, troche, disc, tablet, soft candy, pastille, sub-lingual or buccal tablet.

4. The method of claim 1 wherein the amount of magnesium is between 0.001 and 1000 mg per lozenge.

5. The method of claim 1 wherein the amount of magnesium is between 1 and 500 mg per lozenge.

6. The method of claim 1 wherein the amount of magnesium is between 50 and 200 mg per lozenge.

7. The method of claim 1 wherein the amount of magnesium is about 25 to about 100 mg per lozenge.

8. The method of claim 1 wherein the lozenge is retained in the mouth until it dissolves.

9. The method of claim 1 wherein the lozenge is retained in the mouth for at least 5 minutes.

10. The method of treating asthma and chest congestion in people in need of treatment with orally-retained liquids comprising pharmaceutically acceptable magnesium applied to the mouth and throat in a sustained manner.

11. The method of claim 10 wherein the magnesium compound is selected from the group comprising magnesium chloride, magnesium acetate, magnesium gluconate, magnesium lactate, magnesium malate, magnesium glutamate, magnesium aspartate, magnesium citrate, magnesium glycinate, magnesium carbonate, magnesium hydroxide, magnesium oxide, magnesium carbonate, magnesium phosphate, magnesium orotate, magnesium taurinate, magnesium lysinate, magnesium adipate, magnesium bicarbonate and magnesium sulfate and combinations of said magnesium compounds.

12. The method of claim 10 wherein the liquid comprises a syrup, throat spray, mouth wash, drops or gargle.

13. The method of claim 10 wherein the amount of magnesium is between 0.001 and 1000 mg.

14. The method of claim 10 wherein the amount of magnesium is between 1 and 500 mg.

15. The method of claim 10 wherein the amount of magnesium is between 50 and 200 mg.

16. The method of claim 10 wherein the amount of magnesium is about 25 to about 100 mg.

17. The method of claim 10 wherein the liquid is retained in the mouth for at least 5 minutes.

18. The method of preventing asthma and chest congestion in people in need of treatment with throat lozenges comprising pharmaceutically acceptable magnesium.

19. The method of claim 18 wherein the magnesium compound is selected from the group comprising magnesium chloride, magnesium acetate, magnesium gluconate, magnesium lactate, magnesium malate, magnesium glutamate, magnesium aspartate, magnesium citrate, magnesium glycinate, magnesium carbonate, magnesium hydroxide, magnesium oxide, magnesium carbonate, magnesium phosphate, magnesium orotate, magnesium taurinate, magnesium lysinate, magnesium adipate, magnesium bicarbonate and magnesium sulfate and combinations of said magnesium compounds.

20. The method of claim 18 wherein the lozenge is a compressed tablet, hard candy, troche, disc, tablet, soft candy, pastille, sub-lingual or buccal tablet.

21. The method of claim 18 wherein the amount of magnesium is between 0.001 and 1000 mg.

22. The method of claim 18 wherein the amount of magnesium is between 1 and 500 mg.

23. The method of claim 18 wherein the amount of magnesium is between 50 and 200 mg.

24. The method of claim 18 wherein the amount of magnesium is about 25 to about 100 mg.

25. The method of claim 18 wherein the lozenge is retained in the mouth until it dissolves.

26. The method of claim 18 wherein the lozenge is retained in the mouth for at least 5 minutes.

27. The method of preventing asthma and chest congestion in people in need of treatment with orally-retained liquids comprising pharmaceutically acceptable magnesium applied to the mouth and throat in a sustained manner.

28. The method of claim 27 wherein the magnesium compound is selected from the group comprising magnesium chloride, magnesium acetate, magnesium gluconate, magnesium lactate, magnesium malate, magnesium glutamate, magnesium aspartate, magnesium citrate, magnesium glycinate, magnesium carbonate, magnesium hydroxide, magnesium oxide, magnesium carbonate, magnesium phosphate, magnesium orotate, magnesium taurinate, magnesium lysinate, magnesium adipate, magnesium bicarbonate and magnesium sulfate and combinations of said magnesium compounds.

29. The method of claim 27 wherein the liquid comprises a syrup, throat spray, mouth wash, drops or gargle.

30. The method of claim 27 wherein the amount of magnesium is between 0.001 and 1000 mg.

31. The method of claim 27 wherein the amount of magnesium is between 1 and 500 mg.

32. The method of claim 27 wherein the amount of magnesium is between 50 and 200 mg.

33. The method of claim 27 wherein the amount of magnesium is about 25 to about 100 mg.

34. The method of claim 27 wherein the liquid is retained in the mouth for at least 5 minutes.
